(19) United States
(12) Patent Application Publication
(10) Pub. No.: US 2012/0011599 A1
(43) Pub. Date: Jan. 12, 2012

(54) HYDDROPEROXIDE GENES AND TOLERANCE TO ABIOTIC STRESS IN PLANTS

(75) Inventors: Katayoon Dehesh, Davis, CA (US); Tatyana Savchenko, Davis, CA (US)

(73) Assignee: The Regents of The University of California, Oakland, CA (US)

(21) Appl. No.: 13/203,965

(22) PCT Filed: Mar. 2, 2010
(86) PCT No.: PCT/US2010/025875

§ 371 Date: Aug. 30, 2011

Related U.S. Application Data
(60) Provisional application No. 61/209,064, filed on Mar. 2, 2009.

(51) Int. Cl. A01H 5/00; A01H 5/10; A01H 1/06

(52) U.S. Cl. 800/260; 800/278; 800/293; 800/298; 800/320.2

(57) ABSTRACT

This invention provides for novel methods for preparing a plant tolerant to abiotic stress, such as drought or salt. This invention also provides for transgenic plants and transgenic seeds that are tolerant to abiotic stress. The methods of the present invention comprises introducing a recombinant expression cassette comprising a hydroperoxide lyase polynucleotide encoding a hydroperoxide lyase enzyme into the plants, and selecting a plant that is tolerant to abiotic stress. The transgenic plants and seeds generated by the methods of the invention accordingly comprise a recombinant expression cassette comprising a HPL polynucleotide encoding HPL enzyme.

### STATEMENT AS TO RIGHTS TO INVENTIONS MADE UNDER FEDERALLY SPONSORED RESEARCH AND DEVELOPMENT

This invention was made with government support under grant number 0543904, awarded by the National Science Foundation. The government has certain rights in the invention.

### BACKGROUND OF THE INVENTION

The oxylipin pathway orchestrates a multitude of biological processes in response to developmental and environmental stimuli across the animal and plant kingdoms. The products of the oxylipin pathway are derived from fatty acid oxidation and are designated as oxylipins. In plants, these compounds are mainly derived from the oxidation of α-linolenic (α-LeA: 18:3) and linoleic acids (LA: 18:2).

The biosynthesis of oxylipins is initiated by the action of lipases on complex membrane lipids causing the release of unesterified fatty acids. Subsequently, lipoxygenases (linoleate oxygen oxidoreductases, LOXs) introduce molecular oxygen to either the 9 or the 13 position of 18:2 and 18:3, and convert them into their corresponding 9- or 13-hydroperoxy fatty acids [9/13-hydroperoxyoctadecatrienoic acid (9/13-HPOT) and 9/13-hydroperoxyoctadecadienoic acid (9/13-HPOD)] (Dhondt, S. et al., Plant J. 23:431-440, 2000; Vick, B. A., In: Moore, T. S., Lipid metabolism in plants, CRC Press Inc., Florida, pp. 167-191, 1993; Brash, A. R., J. Biol. Chem. 274:23679-23682, 1999; Narvaez-Vasquez, J. et al., Plant Cell 11:2249-2260, 1999). These hydroperoxides become the substrates for subsequent action of the four major metabolic pathways namely, the peroxygenase (POX), divinyl ether synthase (DES), allene oxide synthase (AOS) and hydroperoxide lyase (HPL) pathways (Feussner, I. and Wasternack, C., Annu. Rev. Plant Physiol. Plant Mol. Biol. 53:275-297, 2002). Among these pathways, the AOS- and HPL-branches are considered to be the two major critical plant stress-response pathways. They compete for the same substrates and are responsible for the production of lipid-based signaling compounds, antimicrobial and antifungal compounds, and aromatic compounds (Feussner, I. and Wasternack, C., supra; Howe, G. and Schilmiller, A. L., Curr. Opin. Plant Bio. 5:230-236, 2002). The AOS branch of 13-LOX transforms 13-HPOT to the jasmonate family of compounds that includes jasmonic acid (JA), methyl jasmonate (MeJA), and their metabolic precursor, 12-oxo-phytodienoic acid (120PDA) (Howe, G. and Schilmiller, A. L., supra).

Though Arabidopsis thaliana has one HPL, many plant species have more than one gene encoding HPL enzymes. For example, Medicago truncatula is reported to have two, and alfalfa and rice each have three HPLs (Noordermeer, M. A. et al., Eur. J. Biochem. 267:2473-2482, 2000; Chehab, E. W. et al., Plant Physiol. 141:121-134, 2006). This variation in the number of genes among plant species may reflect the differential regulation of this pathway and, ultimately, the diversity of the species' responses to various stimuli.

HPL enzymes catalyze the cleavage of 9/13-hydroperoxides and produce a range of metabolites. The action of HPL on 9-HPOT/HPOD gives rise to the bactericidal C9 aldehydes and oxoacids involved in the flavors and odors of fruits and leaves (Vick, B. A., In: Moore, T. S., Lipid metabolism in plants, CRC Press Inc., Florida, pp. 167-191, 1993; Brash, A. R., J. Biol. Chem. 274:23679-23682, 1999; Matsui, K., Curr. Opin. Plant Biol. 9(3):274-280, 2006; Cho, M. J. et al., J. Food Prot. 67:1014-1016, 2004). Activity of HPL on 13-HPOT/HPOD leads to the production of the green leaf volatiles (GLVs) that are comprised of Z-3-hexenal and n-hexanal, and their corresponding alcohols, generated through the action of alcohol dehydrogenase (ADH), and esters, respectively (Matsui, K., Curr. Opin. Plant Biol. 9(3):274-280, 2006). An acyl-transferase (CHAT) converts Z-3-hexenol to Z-3-hexenyl acetate (d'Auria, J. C. et al., Plant J. 49:194-207, 2006). In addition, isomerization of Z-3-hexenal results in generation of E-2-hexenal.

It has been shown that the presence of three rice HPL genes (HPL1, HPL2, and HPL3) are distinct in their levels and patterns of expression (Chehab, E. W. et al., Plant Physiol. 141(1):121-34, 2006). The three corresponding encoded enzymes also differ in their substrate specificity as determined by in vitro enzyme assays, in conjunction with the respective profiles of their cognate metabolites in transgenic Arabidopsis generated in the Columbia-0 ecotype (Col-0) background. The Col-0 ecotype is a natural hpl mutant that expresses the gene transcript but because of a 10 base pair deletion encodes a dysfunctional enzyme and thus lacks C6-aldehydes (Duan, H. et al., Plant Physiol. 139:1529-1544, 2005).

The role of aldehydes generated by overexpression of rice HPL3 in various backgrounds has been examined (Chehab, E. W. et al., PLoS ONE 3(4): e1904, 2008). It has been shown that hexenyl acetate is the predominant wound-inducible volatile signal that mediates indirect defense responses by directing tritrophic (plant-herbivore-natural enemy) interactions.

However, the role of these metabolic pathways, and of the hydroperoxide lyases in particular, on plant stress-responses besides responses to wounding and insect damage is not well understood in the prior art. This and other problems are addressed by the present invention.

### BRIEF SUMMARY OF THE INVENTION

The present invention relates to the development of abiotic stress-tolerant plants. In accordance with various embodiments of the invention, methods of preparing plants with increased abiotic stress-tolerance and/or other advantageous characteristics—such as, for example, increased biomass, increased seed yield, heavier grains, a longer grain-filling period, and/or sturdier stems—are provided. In accordance with an exemplary embodiment, this invention is directed to the preparation of transgenic plants that express a hydroperoxide lyase sequence, and preferably, a heterologous hydroperoxide lyase sequence.

The methods of the invention comprise introducing into a population of plants a recombinant expression cassette comprising a hydroperoxide lyase (HPL) polynucleotide encoding a HPL enzyme; and selecting a plant that is tolerant to abiotic stress, wherein the HPL enzyme comprises (L/I)-(F/C)-G-(Y/F)-(Q/R)-(P/K), wherein the HPL enzyme further comprises (N/D)-K-(Q/I)-C-(A/P)-(G/A)-K-(D/N). The step of introducing the expression cassette can be carried out using any known method. For example, the expression cassette can be introduced by Agrobacterium-mediated transformation of plant cells, a sexual cross or using micro-projectile bombardment of plant cells.

In accordance with one aspect of an exemplary embodiment of the invention, the HPL enzyme is localized outside the plastid when expressed in the population of plants. In some embodiments of the invention, the HPL enzyme recognizes 9-hydroperoxy-octadecatrienoic acid (9-HPOT) or 9-hydroperoxy-octadecadienoic acid (9-HPOD). In some embodiments of the invention, the HPL enzyme recognizes 13-hydroperoxy-octadecatrienoic acid (13-HPOT) or 13-hydroperoxy-octadecadienoic acid (13-HPOD).

In some embodiments of the invention, the HPL enzyme is localized outside the plastid when expressed in the population of plants, and wherein the HPL enzyme recognizes 9-hydroperoxy-octadecatrienoic acid (9-HPOT) or 9-hydroperoxy-octadecadienoic acid (9-HPOD). In some embodiments of the invention, the HPL enzyme further recognizes 13-hydroperoxy-octadecatrienoic acid (13-HPOT) or 13-hydroperoxy-octadecadienoic acid (13-HPOD).

In accordance with one exemplary embodiment of the invention, a method of preparing a plant tolerant to abiotic stress comprises introducing into a population of plants a recombinant expression cassette comprising a hydroperoxide lyase (HPL) polynucleotide encoding a HPL enzyme wherein the HPL enzyme has an amino acid sequence at least 90% identical to SEQ ID NO. 2, 4 or 6, and selecting a plant that is tolerant to abiotic stress, wherein the HPL enzyme is localized extraplastidially when expressed in the population of plants, and wherein the HPL enzyme recognizes 9-hydroperoxy-octadecatrienoic acid (9-HPOT) or 9-hydroperoxy-octadecadienoic acid (9-HPOD), and further recognizes 13-hydroperoxy-octadecatrienoic acid (13-HPOT) or 13-hydroperoxy-octadecadienoic acid (13-HPOD).

In some embodiments of the invention, the abiotic stress is drought. In some embodiments of the invention, the abiotic stress is salinity.

In some embodiments of the invention, the HPL polynucleotide is operably linked to a promoter. The promoter of choice could be either a constitutive promoter, or an inducible promoter, or a tissue-preferred promoter.

In some embodiments of the invention, the HPL enzyme has an amino acid sequence at least 90% identical to SEQ ID NO. 2, 4 or 6. In some embodiments of the invention, the HPL enzyme has an amino acid sequence at least 91%, 92%, 93%, 94%, or 95% identical to SEQ ID NO. 2, 4 or 6. In some embodiments of the invention, the HPL enzyme has an amino acid sequence at least 96%, 97%, 98%, or 99% identical to SEQ ID NO. 2, 4 or 6. In some embodiments of the invention, the amino acid sequence identity of the HPL enzyme to SEQ ID NO. 2, 4 or 6 may be lower than 90% provided that the HPL enzyme comprises (L/I)-(F/C)-G-(Y/F)-(Q/R)-(P/K) and (N/D)-K-(Q/I)-C-(A/P)-(G/A)-K-(D/N). In some embodiments of the invention, the HPL polynucleotide is SEQ ID NO. 1, 3 or 5.

The HPL polynucleotide can be introduced into any plant capable of transformation with recombinant expression constructs. The expression in Oryza sativa is preferred herein. In accordance with various exemplary embodiments of this invention, other dicots or monocots may be utilized with comparable utility.

The present invention also relates to abiotic stress-tolerant transgenic plants. The transgenic plants of the invention have increased abiotic stress-tolerance and/or other advantageous characteristics, such as, for example, increased biomass, increased seed yield, heavier grains, a longer grain-filling period, and/or sturdier stems. This invention is directed to transgenic plants that express a hydroperoxide lyase.

The transgenic plants of the invention comprise a recombinant expression cassette comprising a HPL polynucleotide encoding a HPL enzyme or any active fragment thereof having an amino acid sequence either identical to SEQ ID NO. 2, 4 or 6 or with sufficient identity to SEQ ID NO. 2, 4 or 6 to achieve similar functionality as SEQ ID NO. 2, 4 or 6, wherein the transgenic plants are not Arabidopsis. An example of such transgenic plants is Oryza sativa.

The present invention also relates to transgenic seeds from the transgenic plants of the invention. An example of such transgenic seeds is a transgenic Oryza sativa seed from the transgenic plants of the present invention.

### BRIEF DESCRIPTION OF THE DRAWINGS

FIG. 1: Survival test of HPL1 and HPL2 lines exposed to 200 mM salt. Data are shown as the mean±the standard deviation.

FIG. 2: Survival test of HPL lines exposed to drought. The lines overexpressing HPL1 through 3 are designated as OsHPL1 OE, OsHPL2 OE and OsHPL3 OE, respectively; the line overexpressing HP-3 minus the first 15 amino acids of the plastid transit peptide at the amino terminus of the enzyme is designated as OsHPL3-TP OE. Data are shown as the mean±the standard deviation.

### DETAILED DESCRIPTION OF THE INVENTION

### I. Definitions

The term “HPL polynucleotide” refers to a polynucleotide that is derived from the gene encoding the hydroperoxide lyase polypeptide and encodes a polypeptide that retains hydroperoxide lyase enzymatic activity. HPL encodes hydroperoxide lyase. Several HPL genes have been isolated from rice including, HPL1, HPL2, and HPL3. The term as used herein encompasses a polynucleotide including a native hydroperoxide lyase sequence, as well as modifications and fragments thereof. The term HPL polynucleotide as used herein encompass a polynucleotide including, respectively, a native hydroperoxide lyase sequence as well as modifications and fragments that code for an active HPL polypeptide.

The term “HPOT” refers to hydroperoxy-octadecatrienoic acid. The term “HPOD” refers to hydroperoxy-octadecadienoic acid. The term “9-HPOT” refers to 9-hydroperoxy-octadecatrienoic acid. The term “9-HPOD” refers to 9-hydroperoxy-octadecadienoic acid. The term “13-HPOT” refers to 13-hydroperoxy-octadecatrienoic acid. The term “13-HPOD” refers to 13-hydroperoxy-octadecadienoic acid.

The term “polypeptide” refers to a polymer of amino acids and can include full-length proteins, polypeptide, and fragments thereof. In the present invention, “HPL polypeptide” means a polypeptide having at least one HPL function.

Thus, the term “HPL polynucleotide” and “HPL polypeptide” of the invention may include alterations to the polynucleotide or polypeptide sequences, so long as the alteration results in a molecule displaying HPL activity. Thus, the polynucleotide or polypeptide may be substantially identical to a reference sequence (e.g., SEQ ID NOs: 1-6). The sequence identity may be lower than 90% provided that the HPL enzyme comprises (L/I)-(F/C)-G-(Y/F)-(Q/R)-(P/K) and (N/D)-K-(Q/I)-C-(A/P)-(G/A)-K-(D/N). Whereas some native HPL molecules are localized in the plastid, some are localized outside the plastid. A good way to localize a polypeptide that would normally be localized inside the plastid extraplastidially is to remove the first 15 amino acids of its plastid transit peptide or to fuse it at the amino terminal to another protein and confirm that it is not localized to the plastid. Removal of the transit peptide should not affect enzyme activity; however, the activity displayed by some mutant molecules may not at the same level as the native molecule. Modifications of the polynucleotide sequences described herein typically include deletions, additions and substitutions, to the native HPL sequences. These modifications may be deliberate, as through site-directed mutagenesis, or may be accidental, such as through mutations of plants that express the polynucleotide or errors due to PCR amplification. The term encompasses expressed allelic variants of the wild-type sequence which may occur by normal genetic variation or are produced by genetic engineering methods and which result in HPL activity.

The term “heterologous” in reference to a nucleic acid or polynucleotide of the present invention refers to a nucleic acid or polynucleotide that originates from a foreign species, or if from the same species, is altered in some way (e.g., mutated, added in multiple copies, linked to a non-native promoter or enhancer sequence, etc.).

The term “plastids” mean the organelles in plants including but not limited to chloroplasts and chromoplasts.

The term “progeny” refers generally to the offspring of a cross and includes direct F1 progeny, as well as later generations of F2, F3, etc.

The term “introgression” refers generally to the movement of a gene from one species into the gene pool of another by genetic crosses. Generally, this is accomplished by repeated backcrossing of an interspecific hybrid with one of its parents.

As used herein, the term “abiotic stress” or “abiotic stress condition” refers to the exposure of a plant, plant cell, or the like, to a non-living (“abiotic”) physical or chemical agent or condition that has an adverse effect on metabolism, growth, development, propagation and/or survival of the plant (collectively “growth”). Abiotic stress can be imposed on a plant due, for example, to an environmental factor such as excessive or insufficient water (e.g., flooding, drought, dehydration), anaerobic conditions (e.g., a low level of oxygen), abnormal osmotic conditions, salinity or temperature (e.g., hot/heat, cold, freezing, frost), a deficiency of nutrients or exposure to pollutants, or by a hormone, second messenger or other molecule. Anaerobic stress, for example, is due to a reduction in oxygen levels (hypoxia or anoxia) sufficient to produce a stress response. A flooding stress can be due to prolonged or transient immersion of a plant, plant part, tissue or isolated cell in a liquid medium such as occurs during monsoon, wet season, flash flooding or excessive irrigation of plants, or the like. A cold stress or heat stress can occur due to a decrease or increase, respectively, in the temperature from the optimum range of growth temperatures for a particular plant species. Such optimum growth temperature ranges are readily determined or known to those skilled in the art. Dehydration stress can be induced by the loss of water, reduced turgor, or reduced water content of a cell, tissue, organ or whole plant. Drought stress can be induced by or associated with the deprivation of water or reduced supply of water to a cell, tissue, organ or organism. Saline stress (salt-stress) can be associated with or induced by a perturbation in the osmotic potential of the intracellular or extracellular environment of a cell. Osmotic stress also can be associated with or induced by a change, for example, in the concentration of molecules in the intracellular or extracellular environment of a plant cell, particularly where the molecules cannot be partitioned across the plant cell membrane.

A plant's response to abiotic stress includes the production of excess reactive oxygen species (ROS), including singlet oxygen, superoxide, hydrogen peroxide and hydroxyls radicals, which act as signaling molecules and play a role in the initiation of defense mechanisms. ROS are involved in diverse environmental stress in plants. Excessive temperature extremes, water stress, ion imbalances due to salinity, air pollution, and mechanical damage (such as wounding by sucking or chewing insects or breakage due to wind, etc.) lead to chemical signals propagated through ROS. Adaptation to the stress will involve a quenching of ROS signal through on or more anti-oxidant enzymes or compounds, such as superoxide dismutase (SOD), glutathione, ascorbate, carotenoids, and others. When the plants quenching systems are exceeded by the environmental stress, extensive and rapid degeneration reactions can occur through ROS, such as protein denaturation and lipid peroxidation. The improved tolerance to one particular type of abiotic stress, such as drought, may confer a similarly improved tolerance to another, such as high light or heat, when part of the mechanism of improved tolerance includes improved quenching of oxidative or ROS stress.

Plants suffer heat stress when temperatures are hot enough for a long enough period of time to cause irreversible damage to plant function, development and/or yield. Heat stress can have detrimental effects on reproductive development and reduce yield (abnormal biomass and/or fruit and seed). When subjected to extreme heat stress, plants may not survive.

The invention provides a genetically modified plant, which can be a transgenic plant, that is more tolerant to a stress condition than a corresponding reference plant. As used herein, the term “tolerant” when used in reference to a stress condition of a plant, means that the particular plant, when exposed to a stress condition, shows less of an effect, or no effect, in response to the condition as compared to a corresponding reference plant (naturally occurring wild-type plant or a plant not containing a construct of the present invention). As a consequence, a plant encompassed within the present invention shows improved agronomic performance as a result of enhanced abiotic stress tolerance and grows better under more widely varying conditions, such as increased biomass and/or higher yields and/or produces more seeds. Preferably, the transgenic plant is capable of substantially normal growth under environmental conditions where the corresponding reference plant shows reduced growth, yield, metabolism or viability, or increased male or female sterility.

As used herein, the term “drought-tolerance” refers to the more desirable productivity of a plant under conditions of water deficit stress. Water deficit stress develops as the evapotranspiration demand for water exceeds the supply of water. Water deficit stress can be of large or small magnitude (e.g., days or weeks of little or no accessible water), but drought tolerant plants will show better growth and/or recovery from the stress, as compared to drought sensitive plants.

As used herein, the term “water use efficiency” refers to the more desirable productivity of a plant per unit of water applied. The applied water may be the result of precipitation or irrigation.

As used herein, the term “salt-tolerance” refers to the more desirable productivity of a plant under conditions of salinity stress. While for each species, the threshold at which soil and/or water salinity (often expressed as conductivity, or E.C.) differs, a salt-tolerant plant would have a higher salinity threshold before yields decline. Salt-tolerance also refers to the sensitivity of yield to water and/or soil salinity beyond the threshold. So a salt-tolerant plant would show less impact on yield per unit of salinity (E.C.) than a salt-sensitive plant. Salt-tolerance refers to an increased threshold and/or a decreased sensitivity beyond the threshold of yield to salinity.

The term “plant” includes whole plants, shoot vegetative organs/structures (e.g., leaves, stems and tubers), roots, flowers and floral organs/structures (e.g., bracts, sepals, petals, stamens, carpels, anthers and ovules), seed (including embryo, endosperm, and seed coat) and fruit (the mature ovary), plant tissue (e.g., vascular tissue, ground tissue, and the like) and cells (e.g., guard cells, egg cells, trichomes and the like), and progeny of same. The class of plants that can be used in the method of the invention is generally as broad as the class of higher and lower plants amenable to transformation techniques, including angiosperms (monocotyledonous and dicotyledonous plants), gymnosperms, ferns, and multicellular algae. It includes plants of a variety of ploidy levels, including aneuploid, polyploid, diploid, haploid and hemizygous.

As used herein, “transgenic plant” includes reference to a plant that comprises within its genome a heterologous polynucleotide. Generally, the heterologous polynucleotide is stably integrated within the genome such that the polynucleotide is passed on to successive generations. The heterologous polynucleotide may be integrated into the genome alone or as part of a recombinant expression cassette. “Transgenic” is used herein to include any cell, cell line, callus, tissue, plant part or plant, the genotype of which has been altered by the presence of heterologous nucleic acid, including those transgenics initially so altered as well as those created by sexual crosses or asexual propagation from the initial transgenic. The term “transgenic” as used herein does not encompass the alteration of the genome (chromosomal or extra-chromosomal) by conventional plant breeding methods or by naturally occurring events such as random cross-fertilization, non-recombinant viral infection, non-recombinant bacterial transformation, non-recombinant transposition, or spontaneous mutation.

The term “expression cassette” refers to any recombinant expression system for the purpose of expressing a nucleic acid sequence of the invention in vitro or in vivo, constitutively or inducibly, in any cell, including, in addition to plant cells, prokaryotic, yeast, fungal, insect or mammalian cells. The term includes linear and circular expression systems. The term includes all vectors. The cassettes can remain episomal or integrate into the host cell genome. The expression cassettes can have the ability to self-replicate or not (i.e., drive only transient expression in a cell). The term includes recombinant expression cassettes that contain only the minimum elements needed for transcription of the recombinant nucleic acid.

The term “constitutive” or “constitutively” denotes temporal and spatial expression of the polypeptides of the present invention in plants in the methods according to various exemplary embodiments of the invention. The term “constitutive” or “constitutively” means the expression of the polypeptides of the present invention in the tissues of the plant throughout the life of the plant and in particular during its entire vegetative cycle. In some embodiments, the polypeptides of the present invention are expressed constitutively in all plant tissues. In some embodiments, the polypeptides of the present invention are expressed constitutively in the roots, the leaves, the stems, the flowers, and/or the fruits. In other embodiments of the invention, the polypeptides of the present invention are expressed constitutively in the roots, the leaves, and/or the stems.

The term “inducible” or “inducibly” means the polypeptides of the present invention are not expressed, or are expressed at very low levels, in the absence of an inducing agent. The expression of the polypeptides of the present invention is greatly induced in response to an inducing agent.

The term “inducing agent” is used to refer to a chemical, biological or physical agent or environmental condition that effects transcription from an inducible regulatory element. In response to exposure to an inducing agent, transcription from the inducible regulatory element generally is initiated de novo or is increased above a basal or constitutive level of expression. Such induction can be identified using the methods disclosed herein, including detecting an increased level of RNA transcribed from a nucleotide sequence operatively linked to the regulatory element, increased expression of a polypeptide encoded by the nucleotide sequence, or a phenotype conferred by expression of the encoded polypeptide.

The term “homolog” is used to refer to a gene that is similar in structure and evolutionary origin to a gene in another species. In the case of HPL genes, homologs encode proteins that belong to the cytochrome P450 family and contain the typical signature domains designated as I-, K-helices and the Heme-binding domain, and that catalyze the cleavage of 9/13-hydroperoxides to produce the corresponding metabolites, including, but not limited to, C9 aldehydes and oxoacids from 9-hydroperoxy-octadecatrienoic acids/hydroperoxy-octadecadienoic acids and C8 aldehydes, hexenals and hexanals, from 13-hydroperoxy-octadecatrienoic acids/hydroperoxy-octadecadienoic acids. See Chehab et al. (J. Integrative Plant Biol. 49(1):43-51, 2007) for a description of the phylogenetic analysis and sequence alignments of HPL homologs from several species showing the HPL consensus sequences (L/I)-(F/C)-G-(Y/F)-(Q/R)-(P/K) and (N/D)-K-(Q/I)-C-(A/P)-(G/A)-K-(D/N). As genomic sequences become available, methods known in the art can be used to identify additional HPL homologs from other species.

The phrase “substantially identical,” in the context of the present invention refers to polynucleotides or polypeptides that have sufficient sequence identity with a reference sequence (e.g., one of SEQ ID NOs: 1-6) to effect similar functionality when expressed in plants as the reference sequence. In accordance with one aspect of an exemplary embodiment of the invention, a polynucleotide or a polypeptide that exhibits at least 90% sequence identity with a reference sequence (e.g., one of SEQ ID NOs: 1-6) may be deemed to be “substantially identical;” however, polynucleotides and polypeptides that exhibit less (even significantly less, e.g., 60%-70% or less) than 90% sequence identity may, in accordance with various exemplary embodiments of the invention, be “substantially identical” to their reference sequences if requisite functionality is achieved. Alternatively, percent identity can be any value from 90% to 100%. More preferred embodiments include at least: 90%, 95%, or 99% identity as used herein is as compared to the reference sequence using the programs described herein; preferably BLAST using standard parameters, as described below. The sequence identity of the polynucleotides and plypeptides may be lower than 90% provided that the HPL enzyme comprises (L/I)-(F/C)-G-(Y/F)-(Q/R)-(P/K) and (N/D)-K-(Q/I)-C-(A/P)-(G/A)-K-(D/N).

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters.

A “comparison window,” as used herein, includes reference to a segment of any one of the number of contiguous positions, such as from 20 to 600, usually about 50 to about 200, more usually about 100 to about 150, in which a sequence may be compared to a reference sequence of the same number of contiguous positions after the two sequences are optimally aligned. If no range is provided, the comparison window is the entire length of the reference sequence. Methods of alignment of sequences for comparison are well-known in the art. Optimal alignment of sequences for comparison can be conducted [e.g., by the local homology algorithm of Smith and Waterman, Adv. Appl. Math. 2:482, 1981; by the homology alignment algorithm of Needleman and Wunsch, J. Mol. Biol. 48:443, 1970; by the search for similarity method of Pearson and Lipman, Proc. Nat'l. Acad. Sci. USA 85:2444, 1988; by computerized implementations of these algorithms (GAP, BESTFIT, FASTA, and TFASTA in the Wisconsin Genetics Software Package, Genetics Computer Group, 575 Science Dr., Madison, Wis.), or by manual alignment and visual inspection].

An example of an algorithm that is suitable for determining percent sequence identity and sequence similarity is the BLAST algorithm, which is described in Altschul, S. F. et al., J. Mol. Biol. 215:403-410, 1990. Software for performing BLAST analyses is publicly available through the National Center for Biotechnology Information. This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul, S. F. et al., supra). These initial neighborhood word hits act as seeds for initiating searches to find longer HSPs containing them. The word hits are extended in both directions along each sequence for as far as the cumulative alignment score can be increased. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLAST program uses as defaults a wordlength (W) of 11, the BLOSUM62 scoring matrix (see Henikoff and Henikoff, Proc. Natl. Acad. Sci. USA 89:10915, 1989), alignments (B) of 50, expectation (E) of 10, M=5, N=−4, and a comparison of both strands.

The BLAST algorithm also performs a statistical analysis of the similarity between two sequences (see, e.g., Karlin and Altschul, Proc. Nat'l. Acad. Sci. USA 90:5873-5787, 1993). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.2, preferably less than about 0.01, and more preferably less than about 0.001.

### II. Nucleic Acids

In accordance with one aspect of an exemplary embodiment of the present invention, a polynucleotide may include (a) a polynucleotide encoding a polypeptide of SEQ ID NO: 2, SEQ ID NO: 4 or SEQ ID NO: 6, including exemplary polynucleotides of SEQ ID NO: 1, SEQ ID NO: 3, and SEQ ID NO: 5; (b) a polynucleotide having a specified sequence identity with polynucleotides of (a); (c) homologs of SEQ ID NOs: 1, 3 and 5; (d) complementary sequences of polynucleotides of (a), (b) or (c); and (e) active fragments of any of (a), (b), (c) or (d).

The present invention provides, among other things, isolated nucleic acids of RNA, DNA, and analogs and/or chimeras thereof, comprising a polynucleotide of the present invention.

### A. Polynucleotides Encoding a Polypeptide of the Present Invention

The present invention provides isolated nucleic acids comprising a polynucleotide of the present invention, wherein the polynucleotide encodes a polypeptide of the present invention or an active fragment thereof. Every nucleic acid sequence herein that encodes a polypeptide also, by reference to the genetic code, describes every possible silent variation of the nucleic acid. One of ordinary skill will recognize that each codon in a nucleic acid (except AUG, which is ordinarily the only codon for methionine; and UGG, which is ordinarily the only codon for tryptophan) can be modified to yield a functionally identical molecule. Thus, each silent variation of a nucleic acid, which encodes a polypeptide of the present invention, is implicit in each described polypeptide sequence and is within the scope of the present invention. Accordingly, the present invention includes polynucleotides of the present invention and polynucleotides encoding a polypeptide of the present invention.

B. Polynucleotides Having a Specific Sequence Identity with the Polynucleotides of (A)

In accordance with various exemplary embodiments, the present invention provides isolated HPL nucleic acids comprising HPL polynucleotides as discussed herein above, wherein the HPL polynucleotides have a specified identity at the nucleotide level to a polynucleotide as disclosed above in section (A) above. Percent identity can be calculated using, for example, the BLAST algorithm under default conditions.

### C. Polynucleotides That Are Homologs

The present invention provides isolated HPL nucleic acids comprising HPL nucleotides that are homologs of SEQ ID NOs: 1, 3 and 5. Some HPL homologs are described in Chehab et al. (J. Integrative Plant Biol. 49(1):43-51, 2007), others can be identified using methods known in the art, and as additional genomic sequences become available additional HPL homologs from other species can be identified.

### D. Polynucleotides Complementary to the Polynucleotides of (A)-(C)

The present invention provides isolated nucleic acids comprising polynucleotides complementary to the polynucleotides of sections A-B, above. As those of skill in the art will recognize, complementary sequences base pair throughout the entirety of their length with the polynucleotides of sections (A)-(C) (i.e., sequences that are 100% complementary over their entire length). Complementary bases associate through hydrogen bonding in double stranded nucleic acids. For example, the following base pairs are complementary: guanine and cytosine; adenine and thymine; and adenine and uracil. Moreover, those skilled in the art will recognize that sequences that base pair throughout the entirety of their regions of overlap (i.e., are 100% complementary in overlapping regions, but are not 100% complementary over their entire length) may be complementary. Furthermore, sequences that are not 100% complementary can still work as anti-sense constructs, and thus may achieve the stated function of this aspect of the invention notwithstanding lesser complementarity (e.g., 60%-70% or less).

### III. Construction of Nucleic Acids

The isolated nucleic acids of the present invention can be made using standard recombinant methods, synthetic techniques, combinations thereof, or any other method now known or hereafter developed for preparing such nucleic acids.

### A. Recombinant Methods for Constructing Nucleic Acids

The isolated nucleic acid compositions of this invention can be obtained from plant biological sources (e.g., tissues from the plant) using any number of cloning methodologies now known to or hereafter devised by those of skill in the art. In some embodiments, oligonucleotide probes that selectively hybridize under stringent conditions to the polynucleotides of the present invention are used to identify the desired sequence in a cDNA or genomic DNA library. Isolation of RNA and construction of cDNA and genomic libraries is well known to those of ordinary skill in the art. See, e.g., Plant Molecular Biology: A Laboratory Manual, Clark, Ed., Springer-Verlag, Berlin, 1997; and Current Protocols in Molecular Biology, Ausubel, et al., Eds., Greene Publishing and Wiley-Interscience, New York, 1995.

### A1. Genomic DNA

The isolated nucleic acid compositions of this invention can be obtained directly from genomic DNA isolated from Oryza sativa (Chehab, et al., Plant Phys. 141: 121-134, 2006).

A2. cDNA Libraries

A number of cDNA synthesis protocols have been described that provide enriched full-length cDNA libraries. Enriched full-length cDNA libraries are constructed to comprise at least 60%, and more preferably at least 70%, 80%, 90% or 95% full-length inserts amongst clones containing inserts. The length of insert in such libraries can be at least 2, 3, 4, 5, 6, 7, 8, 9, 10 or more kilobase (kb) pairs. Vectors to accommodate inserts of these sizes are known in the art and available commercially. See, e.g., Stratagene's lambda ZAP Express (cDNA cloning vector with 0 to 12 kb cloning capacity). An exemplary method of constructing a greater than 95% pure full-length cDNA library is described by Carninci et al., Genomics 37:327-336, 1996. Other methods for producing full-length libraries are known in the art. See, e.g., Edery et al., Mol. Cell Biol. 15(6):3363-3371, 1995; and PCT Application WO/1996/034981.

A non-normalized or subtracted cDNA library also can be used for constructing nucleic acids of the present invention according to standard protocols.

The cDNA or genomic library can be screened using a probe based upon the sequence of a HPL polynucleotide of the present invention such as those disclosed herein. Probes may be used to hybridize with genomic DNA or cDNA sequences to isolate homologous genes in the same or different plant species. Those of skill in the art will appreciate that various degrees of stringency of hybridization can be employed in the assay; and either the hybridization or the wash medium can be stringent.

The nucleic acids of interest can also be amplified from nucleic acid samples using amplification techniques. For instance, polymerase chain reaction (PCR) technology can be used to amplify the sequences of polynucleotides of the present invention and related genes directly from genomic DNA or cDNA libraries. PCR and other in vitro amplification methods may also be useful, for example, to clone nucleic acid sequences that code for proteins to be expressed, to make nucleic acids to use as probes for detecting the presence of the desired mRNA in samples, for nucleic acid sequencing, or for other purposes. The T4 gene 32 protein (Boehringer Mannheim) can be used to improve yield of long PCR products.

PCR-based screening methods have been described. Wilfinger et al. describe a PCR-based method in which the longest cDNA is identified in the first step so that incomplete clones can be eliminated from study (Bio Techniques 22(3): 481-486, 1997). Such methods are particularly effective in combination with a full-length cDNA construction methodology, above.

### B. Synthetic Methods for Constructing Nucleic Acids

The isolated nucleic acids of the present invention also can be prepared by direct chemical synthesis by methods such as the phosphotriester method of Narang et al., Meth. Enzymol. 68: 90-99, 1979; the phosphodiester method of Brown et al., Meth. Enzymol. 68: 109-151, 1979; the diethylphosphoramidite method of Beaucage et al., Tetra. Letts. 22: 1859-1862, 1981; the solid phase phosphoramidite triester method described by Beaucage and Caruthers, Tetra. Letts. 22(20): 1859-1862, 1981; e.g., using an automated synthesizer, e.g., as described in Needham-VanDevanter et al., Nucleic Acids Res. 12: 6159-6168, 1984; and, the solid support method of U.S. Pat. No. 4,458,066. Chemical synthesis generally produces a single stranded oligonucleotide. This may be converted into double stranded DNA by hybridization with a complementary sequence, or by polymerization with a DNA polymerase using the single strand as a template. One of skill will recognize that while chemical synthesis of DNA is best employed for sequences of about 100 bases or less, longer sequences may be obtained by the ligation of shorter sequences.

### IV. Recombinant Expression Cassettes

In accordance with another aspect of an exemplary embodiment, the present invention provides recombinant expression cassettes comprising a nucleic acid of the present invention. A nucleic acid sequence coding for the desired polynucleotide of the present invention, for example a cDNA or a genomic sequence encoding a full length polypeptide of the present invention, can be used to construct a recombinant expression cassette, which can be introduced into the desired host cell. A recombinant expression cassette will typically comprise a polynucleotide of the present invention operably linked to transcriptional initiation regulatory sequences, which will direct the transcription of the polynucleotide in the intended host cell, such as tissues of a transformed plant.

For example, plant expression vectors may include (1) a cloned plant gene under the transcriptional control of 5′ and 3′ regulatory sequences and (2) a dominant selectable marker. Such plant expression vectors may also contain, if desired, a promoter regulatory region (e.g., one conferring inducible or constitutive, environmentally- or developmentally-regulated, or cell- or tissue-specific/selective expression), a transcription initiation start site, a ribosome binding site, an RNA processing signal, a transcription termination site, and/or a polyadenylation signal.

### A. Vectors

Typical vectors useful for expression of genes in higher plants are well known in the art. A number of expression vectors suitable for stable transformation of plant cells or for the establishment of transgenic plants have been described including those described in Weissbach and Weissbach, Methods for Plant Molecular Biology, Academic Press, 1989, and Gelvin et al., Plant Molecular Biology Manual, Kluwer Academic Publishers, 1990. Specific examples include those derived from a tumor-inducing (Ti) plasmid or a root-inducing (Ri) plasmid of Agrobacterium tumefaciens, as well as those disclosed by Herrera-Estrella, L., et al. (Nature 303:209, 1983), Bevan, M. (Nucl. Acids Res. 12: 8711-8721, 1984) and Klee, H. J. (Bio/Technology 3:637-642, 1985) for dicotyledonous plants. Ti-derived plasmids can be transferred into both monocotonous and docotyledonous species using Agrobacterium-mediated transformation (Ishida et al., Nat. Biotechnol. 14:745-50, 1996; Barton et al., Cell 32:1033-1043, 1983). Exemplary Agrobacterium tumefaciens vectors useful herein are plasmids pKYLX6 and pKYLX7 of Schardl, et al. (Gene 61:1-11, 1987) and Berger et al. (Proc. Natl. Acad. Sci. USA 86:8402-6, 1989). Another useful vector herein is plasmid pBI101.2 that is available from CLONTECH Laboratories, Inc. (Palo Alto, Calif.).

Alternatively, non-Ti vectors can be used to transfer the DNA into plants and cells by using free DNA delivery techniques. Such methods may involve, for example, the use of liposomes, electroporation, microprojectile bombardment, silicon carbide whiskers, and viruses. An immature embryo can also be a good target tissue for direct DNA delivery techniques by using the particle gun (Weeks, T. et al., Plant Physiol. 102:1077-1084, 1993; Vasil, V., Bio/Technology 10:667-674, 1993; Wan, Y. and Lemeaux, P., Plant Physiol. 104:37-48, 1994) and for Agrobacterium-mediated DNA transfer (Ishida et al., Nature Biotech. 14:745-750, 1996).

### B. Promoters

### B1. Constitutive Promoters

A number of promoters can be used in the practice of the invention. A plant promoter fragment can be employed which will direct expression of a polynucleotide of the present invention in all tissues of a regenerated plant. Such promoters are referred to herein as “constitutive” promoters and are active under most environmental conditions and state of development or cell differentiation. Examples of constitutive promoters include the cauliflower mosaic virus (CaMV) 35S transcription initiation region.

### B2. Inducible Promoters

Alternatively, the plant promoter can direct expression of a polynucleotide of the present invention under environmental control. Such promoters are referred to here as “inducible” promoters. Environmental conditions that may effect transcription by inducible promoters include biotic stress, abiotic stress, saline stress, drought stress, pathogen attack, anaerobic conditions, cold stress, heat stress, hypoxia stress or the presence of light.

Examples of inducible promoters include, but are not limited to, a salt-inducible promoter rd29A (Kasuga, M. et al., Nature Biotechnol. 17, 287-291, 1999), the drought-inducible promoter of maize (Busk et al., Plant J. 11:1285-1295, 1997); the cold, drought, and high salt inducible promoter from potato (Kirch, Plant Mol. Biol. 33:897-909, 1997), a light-inducible promoter PPDK, a light-inducible promoter from the small subunit of ribulose-1,5-bis-phosphate carboxylase (ssRUBISCO), a hypoxia or cold stress-inducible promoter Adh1, a heat stress-inducible promoter Hsp70 promoter, and many cold inducible promoters known in the art, for example rd29a and cor15a promoters from Arabidopsis thaliana (GenBank ID: D13044 and U01377), blt1O1 and blt4.8 from barley (GenBank ID: AJ310994 and U63993), wcs120 from wheat (GenBank ID: AF031235), and mlipl5 from corn (GenBank ID: D26563).

Other inducible promoters that have been described include the ABA- and turgor-inducible promoters, the promoter of the auxin-binding protein gene (Schwob et al., Plant J. 4(3):423-432, 1993), the UDP glucose flavonoid glycosyl-transferase gene promoter (Ralston et al., Genetics 119:185-197, 1988), the MPI proteinase inhibitor promoter (Cordero et al., Plant J. 6(2):141-150, 1994), and the glyceraldehyde-3-phosphate dehydrogenase gene promoter (Kohler et al., Plant Mol. Biol. 29(6):1293-1298, 1995; Quigley et al., J. Mol. Evol. 29(5):412-421, 1989; Martinez et al., J. Mol. Biol. 208(4):551-565, 1989).

### B3. Tissue-Preferred Promoters

Examples of promoters under developmental control include promoters that initiate transcription only, or preferentially, in certain tissues, such as leaves, roots, fruit, seeds, or flowers. These promoters are sometimes called tissue-preferred promoters. Exemplary promoters include the anther specific promoter 5126 (U.S. Pat. Nos. 5,689,049 and 5,689,051), glob-1 promoter, and gamma-zein promoter. An exemplary promoter for leaf- and stalk-preferred expression is MS8-15 (PCT Publication No. WO 98/00533). Examples of seed-preferred promoters included, but are not limited to, 27 kD gamma zein promoter and waxy promoter (Boronat, A. et al., Plant Sci. 47:95-102, 1986; Reina, M. et al, Nucleic Acids Res. 18(21):6426, 1990; and Kloesgen, R. B. et al., Mol. Gen. Genet. 203:237-244, 1986). Promoters that express in the embryo, pericarp, and endosperm are disclosed in PCT Publication Nos. WO 00/11177 and WO 00/12733 both of which are hereby incorporated by reference. The operation of a promoter may also vary depending on its location in the genome. Thus, a developmentally regulated promoter may become fully or partially constitutive in certain locations. A developmentally regulated promoter can also be modified, if necessary, for weak expression.

Both heterologous and non-heterologous (i.e., endogenous) promoters can be employed to direct expression of the nucleic acids of the present invention. These promoters can also be used, for example, in recombinant expression cassettes to drive expression of antisense nucleic acids to reduce, increase, or alter concentration and/or composition of the proteins of the present invention in a desired tissue. Thus, in some embodiments, the nucleic acid construct will comprise a promoter functional in a plant cell, such as in Zea mays, operably linked to a polynucleotide of the present invention. Promoters useful in these embodiments include the endogenous promoters driving expression of a polypeptide of the present invention.

In some embodiments, isolated nucleic acids which serve as promoter or enhancer elements can be introduced in the appropriate position (generally upstream) of a non-heterologous form of a polynucleotide of the present invention so as to up or down regulate expression of a polynucleotide of the present invention. For example, endogenous promoters can be altered in vivo by mutation, deletion, and/or substitution (see, Kmiec, U.S. Pat. No. 5,565,350 and Zarling et al., U.S. Pat. No. 5,763,240), or isolated promoters can be introduced into a plant cell in the proper orientation and distance from a gene of the present invention so as to control the expression of the gene. Gene expression can be modulated under conditions suitable for plant growth so as to alter the total concentration and/or alter the composition of the polypeptides of the present invention in plant cell. Thus, the present invention provides compositions, and methods for making, heterologous promoters and/or enhancers operably linked to a native, endogenous (i.e., non-heterologous) form of a polynucleotide of the present invention.

In accordance with other exemplary embodiments, the expression cassettes of the present invention may further include an enhancer element, a polyadenylation region, an intron enhancement element, a selectable marker, and/or a terminator element.

The expression cassettes of the invention can be used to confer abiotic stress-tolerance on essentially any plant. In particular, the invention has use in monocots, such as cereal plants, for example, from the genera Avena, Hordeum, Oryza, Secale, Sorghum, Triticum, and Zea. The invention also has use over a broad range of plants, including species from the genera Asparagus, Atropa, Brassica, Citrus, Citrullus, Capsicum, Cucumis, Cucurbita, Daucus, Fragaria, Glycine, Gossypium, Helianthus, Heterocallis, Hyoscyamus, Lactuca, Linum, Lolium, Lycopersicon, Malus, Manihot, Majorana, Medicago, Nicotiana, Panieum, Pannesetum, Persea, Pisum, Pyrus, Prunus, Raphanus, Senecio, Sinapis, Solanum, Trigonella, Vitis, and Vigna. In some embodiments of the invention, the expression cassettes of the invention are used to confer drought-tolerance. In some embodiments of the invention, the expression cassettes of the invention are used to confer salt-tolerance.

### V. Plant Transformation

Once an expression cassette comprising a polynucleotide of the present invention has been constructed, any technique now known or hereafter devised by those skilled in the art may be used to introduce the polynucleotide into a plant. See, for example, protocols described in Ammirato et al., Handbook of Plant Cell Culture—Crop Species. Macmillan Publ. Co., 1984. Shimamoto et al., Nature 338:274-276, 1989; Fromm et al., Bio/Technology 8:833-839, 1990; and Vasil et al., Bio/Technology 8:429-434, 1990.

Transformation and regeneration of plants is generally known in the art, and the selection of the most appropriate transformation technique for a particular embodiment of the invention may be determined by the practitioner. Suitable methods may include, but are not limited to: electroporation of plant protoplasts; liposome-mediated transformation; polyethylene glycol (PEG) mediated transformation; transformation using viruses; micro-injection of plant cells; micro-projectile bombardment of plant cells; vacuum infiltration; and Agrobacterium tumeficiens mediated transformation. Transformation means introducing a nucleotide sequence in a plant in a manner to cause stable or transient expression of the sequence. Examples of these methods in various plants include: U.S. Pat. Nos. 5,571,706; 5,677,175; 5,510,471; 5,750,386; 5,597,945; 5,589,615; 5,750,871; 5,268,526; 5,780,708; 5,538,880; 5,773,269; 5,736,369 and 5,610,042.

Following transformation, plants preferably are selected using a dominant selectable marker incorporated into the transformation vector. Typically, such a marker will confer antibiotic or herbicide resistance on the transformed plants, and selection of transformants can be accomplished by exposing the plants to appropriate concentrations of the antibiotic or herbicide.

The cells, which have been transformed, may be grown into plants in accordance with conventional ways. See, for example, McCormick et al., Plant Cell Reports 5:81-84, 1986. These plants may then be grown, and either pollinated with the same transformed strain or different strains, and the resulting hybrid having the desired phenotypic characteristic identified. Two or more generations may be grown to ensure that the subject phenotypic characteristic is stably maintained and inherited and then seeds harvested to ensure the desired phenotype or other property has been achieved.

### VI. Production of Transgenic Plants by Genetic Crosses

The present invention relates to methods of generating abiotic stress-tolerant plants by transferring a nucleic acid of the present invention, from a donor plant into a recipient plant strain which is not abiotic stress-tolerant, thus conferring the trait of abiotic stress-tolerance to the recipient strain.

Accordingly, one method to accomplish such a transfer is by introgression of a nucleic acid sequence conferring or contributing to this trait from an abiotic stress-tolerant donor plant into a recipient plant that is not abiotic stress-tolerant by crossing said plants. This transfer may thus suitably be accomplished by using traditional breeding techniques.

In one method, a donor plant that exhibits abiotic stress-tolerance and comprising a nucleic acid of the present invention, is crossed with a plant that is not abiotic stress-tolerant and preferably exhibits commercially desirable characteristics, such as, heavier grains, a longer grain-filling period, and sturdier stems, etc. The resulting plant population (representing the F1 hybrids) is then self-pollinated and seeds are obtained (F2 seeds). The F2 seeds can then be screened for abiotic stress-tolerance as by any of the methods described herein.

Inbred abiotic stress-tolerant plant lines can be developed using the techniques of recurrent selection and backcrossing, selfing and/or dihaploids or any other technique used to make parental lines. In a method of selection and backcrossing, abiotic stress-tolerance trait can be introgressed into a target recipient plant (which is called the recurrent parent) by crossing the recurrent parent with a first donor plant (which is different from the recurrent parent and referred to herein as the “non-recurrent parent”). The recurrent parent is a plant that is not abiotic stress-tolerant and possesses commercially desirable characteristics.

The non-recurrent parent exhibits abiotic stress-tolerance and comprising a nucleic acid of the present invention, wherein the expression of the polypeptides of the present invention is enhanced as compared to a plant that is not abiotic stress-tolerant. The non-recurrent parent can be any plant variety or inbred line that is cross-fertile with the recurrent parent. The progeny resulting from a cross between the recurrent parent and non-recurrent parent are backcrossed to the recurrent parent. The resulting plant population is then screened. F1 hybrid plants that comprise the requisite nucleic acid of the present invention are then selected and selfed and selected for a number of generations in order to allow for the plant to become increasingly inbred. This process of continued selfing and selection can be performed for two to five or more generations. The result of such breeding and selection is the production of lines that are genetically homogenous for the genes associated with abiotic stress-tolerance as well as other genes associated with traits of commercial interest.

Abiotic stress-tolerance can be assayed according to any of a number of well-know techniques. The determination that a plant modified according to a method of the invention has increased tolerance to a stress-inducing condition can be made by comparing the treated plant with a control (reference) plant using well-known methods. For example, a plant having increased tolerance to salt stress can be identified by growing the plant on a medium such as soil that contains salt at a level more than about 100% of the amount of salt in the medium on which the corresponding reference plant is capable of growing. Advantageously, a plant treated according to a method of the invention can grow on a medium or soil containing salt at a level of at least about 110%, preferably at least about 150%, more preferably at least about 200%, and optimally at least about 400% of the level of salt in the medium or soil on which a corresponding reference plant can grow. In particular, such a treated plant can grow on medium or soil containing at least 40 mM, generally at least 100 mM, particularly at least 200 mM, and preferably at least 300 mM salt, including, for example, a water soluble inorganic salt such as sodium sulfate, magnesium sulfate, calcium sulfate, sodium chloride, magnesium chloride, calcium chloride, potassium chloride, or the like; salts of agricultural fertilizers, and salts associated with alkaline or acid soil conditions; particularly NaCl.

Drought-tolerance can be determined by any of a number of standard measures including turgor pressure, growth, yield and the like. For example, a plant having increased tolerance to drought can be identified by growing the plant under conditions in which less than the optimal amount of water is provided to the plant through precipitation and/or irrigation. Particularly, a plant having increased tolerance to drought can be identified by growing the plant on a medium such as soil that contains less water than the medium on which the corresponding reference plant is capable of growing. Advantageously, a plant treated according to a method of the invention can grow on a medium or soil containing salt at a level of less than about 90%, preferably less than about 80%, more preferably less than about 50%, and optimally less than about 20% of the amount of water in the medium or soil on which a corresponding reference plant can grow. Alternatively, a plant having increased tolerance to drought can be identified by its ability to recover from drought when rehydration is provided after a period of drought. Advantageously, a plant treated according to a method of the invention can recover when rehydration is provided after a period of at least 3 days drought, at least 5 days drought, preferably at least 7 days drought, more preferably at least about 10 days drought, and optimally at least about 18 days drought.

Water use efficiency can be determined by evaluating the amount of dry biomass that a plant accumulates (which can be vegetative, reproductive, or both, depending on the yield component(s) of interest) per unit water available to the plant. A plant having enhanced water use efficiency will have a greater amount of dry biomass accumulation per unit water available than the corresponding reference plant grown under the same conditions. Water use efficiency at the leaf or plant scale refers to the ratio between the net CO2 assimilation rate and the transpiration rate, usually measured over a period of seconds or minutes. A plant with enhanced water use efficiency will have higher yields (such as 1-5%, 5-10%, 10-15% higher) under restricted water conditions compared to the corresponding reference plant grown under the same conditions.

Heat tolerance can be determined by evaluating the amount of dry biomass that a plant accumulates (which can be vegetative, reproductive, or both, depending on the yield component(s) of interest) relative to increasing temperatures. A plant having enhanced heat tolerance will have higher yields (such as 1-5%, 5-10%, 10-15% higher) under increased temperature conditions (such as 1° C., 2° C., 3° C., 4° C., etc.) compared to the corresponding reference plant grown under the same conditions.

Once the appropriate selections are made, the process is repeated. The process of backcrossing to the recurrent parent and selecting for abiotic stress-tolerance is repeated for approximately five or more generations. The progeny resulting from this process are heterozygous for one or more genes that encode for abiotic stress-tolerance. The last backcross generation is then selfed in order to provide for homozygous pure breeding progeny for abiotic stress-tolerance.

The abiotic stress-tolerant inbred plant lines described herein can be used in additional crossings to create further abiotic stress-tolerant hybrid plants. For example, a first abiotic stress-tolerant inbred plant of the invention can be crossed with a second inbred plant possessing other commercially desirable traits. The second inbred plant line may or may not also display abiotic stress-tolerance.

### VII. Examples

### Example 1

### Cloning and Sequence Analysis of Rice HPLs

Genomic DNA isolated from rice L. cv Nipponbare was used for PCR-based amplification of these genes using the following gene-specific oligonucleotides: OsHPL1 (Forward: 5′-ATAGATATCGCATGCATGGCGCCGCCGCGAGCCAACTCCG-3′ and Reverse: 5′-ATATACGTACTGCAGCGCGCGCCGCCGCTTGACACTATTA-3′), OsHPL2 (Forward: 5′-ATAGATATCGCATGCATGGCGCCACCGCCAGTGAACTCCG-3′ and Reverse: 5′ATATACGTACTGCAGGCACGTGACGTCGACGTGCGTGCTA-3′), and OsHPL3 (Forward: 5′-ATAGATATCGCATGCATGGTGCCGTCGTTCCCGCAGCCGG-3′ and Reverse: 5′-ATATACGTACTGCAGGAGAGAATGGCGGCAGCAAAGCTTA-3′). For each amplification, 30 PCR cycles were carried out using a Gene Amp PCR system 9700 (Applied Biosystems) in a 25 μL reaction mix containing 10 mM Tris-HCl (pH 8.3), 50 mM KCl, 1.5 mm MgCl₂, 4% dimethyl sulfoxide (DMSO), 100 μm of each dNTP, 500 nM of each forward and reverse primer, 0.625 units of Taq DNA polymerase (Invitrogen), and 50 ng of the genomic DNA. Amplification was conducted at 94° C. for 1 min, 94° C. for 30 s, 55° C. for OsHPL1, 63° C. for OsHPL2, and 55° C. for OsHPL3 for 1 mM, 72° C. for 90 s, and a 10-min extension step at 72° C. The amplified products were resolved by electrophoresis on a 1% (w/v) agarose gel. The band corresponding to each full-length gene was cut, purified using QIAquick Gel extraction kit (Qiagen), and cloned in pCR 2.1-TOPO Vector (Invitrogen) according to the manufacturer's instructions. The identities of these clones were confirmed by DNA sequencing. All DNA as well as polypeptide sequence analyses were performed using Vector NTI advance program 9 (Invitrogen).

### Example 2

### Arabidopsis Transformation of Three Rice HPLs

Green fluorescent protein (GFP) fusions for stable expression were constructed by cloning the PCR-amplified, TOPO-cloned, and EcoRI-/BamHI-digested fragments of the full length of all three rice HPLs into the EcoRI/BamHI site of pEZS-NLGFP. Primers were designed to eliminate stop codons and fuse the coding sequences to the 5′ end of the GFP gene. For OsHPL1, the primers used were: Forward: 5′-ATA-GAATTCATGGCGCCGCCGCGAG-3′ and Reverse: 5′-ATAGGATCCGCTA-CTCCGCGCCGCGCG-3′. For OsHPL2, the primers used were: Forward: ATAGAATTCATGGCGCCACCGCCAGT-3′ and Reverse: 5′-ATAGGATCC-GCTCCCGACGACGCCCGT-3′. OsHPL3 was amplified using the following primers: Forward: 5′-ATAGAATTCATGGTGCCGTCGTTCCC-3′ and Reverse: 5′-ATAGGATCCGCGCTGGGAGTGAGCTCCC-3′. To generate OsHPL3-TP (HPL3 minus the first 15 amino acids of the plastid transit peptide at the amino terminus of the protein), OsHPL3 cDNA was amplified (Forward: 5′-CCGGCCAATACCGGGG-3′ and Reverse 5′-TTAGCTGGGAGTGAGCTC-3′). PCR amplifications were conducted as described above with a Tₘ=55° C. used for all genes amplified. GFP fusions for Arabidopsis transformation were created by subcloning the OsHPL1, OsHPL2, and OsHPL3 open reading frames from pEZS-NLGFP into a binary vector using NotI restriction sites with the GFP gene at the C terminus of each gene. For OsHPL3-TP, the PCR product was cloned into Gateway pENTER vector, according to the manufacturer's recommendation. The construct was then fully sequenced, and the pB7WGF2-OsHPL3-15AA TP construct was generated in a recombination reaction between the entry clone pENTR OsHPL3-15AA TP and pB7WGF2 vector. The constructs were verified by sequencing, introduced into Agrobacterium EHA101 strain, and used to transform Arabidopsis plants by using the floral-dip method (Clough, S. J. and Bent, A. F., Plant J. 16: 735-743, 1998). The T1 plants were germinated on soil. Selection of transgenics was by treating 10- to 12-d-old seedlings with 1:1,000 Finale (the commercial product that is 5.78% glufosinate ammonium) twice a week. The localization of OsHPL1, HPL2, and HPL3-TP outside the plastid and OsHPL3 inside the plastid was confirmed in transformed plants.

### Example 3

### Expression of Rice HPL1 and/or HPL2 in Arabidopsis Confers Salt-Tolerance

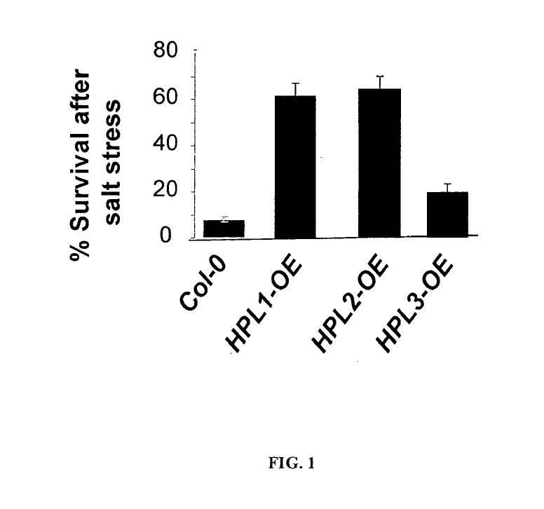

Enhanced tolerance of both HPL1 (p≦0.003) and HPL2 (p≦0.001) lines to salt-stress was observed, as measured by the survival rate of plants exposed to 200 mM NaCl for five days (FIG. 1). Col-0, a natural hpl null mutant, was used as a control. Homozygous lines expressing the corrected version of the HPL genes under endogenous promoter of Col-0 were used.

In a second experiment, five week old HPL2 and Col-0 plants were subjected to salt treatment for three weeks followed by recovery for ten days. Plants were watered one every three days with a nutrient solution (modified Spalding solution). The volume of liquid added was such that it allowed for 1/3 leaching volume. For the pot sizes used, 50-75 ml of nutrient solution was added per pot. When plants were treated with salt, they were watered with the nutrient solution plus 100 mM NaCl once every three days. During the recovery period, plants were watered with the nutrient solution every three days. The HPL2 line had a greater survival rate following 100 mM salt stress than the Col-0 line when plants were grown on either Sunshine Mix #3 (67% versus 50%) or a 50/50 mix of Sunshine Mix #3 and Profile Green (31% versus 21%).

### Example 4

### Expression of Rice HPLs Outside the Plastid in Arabidopsis Confers Drought-Tolerance

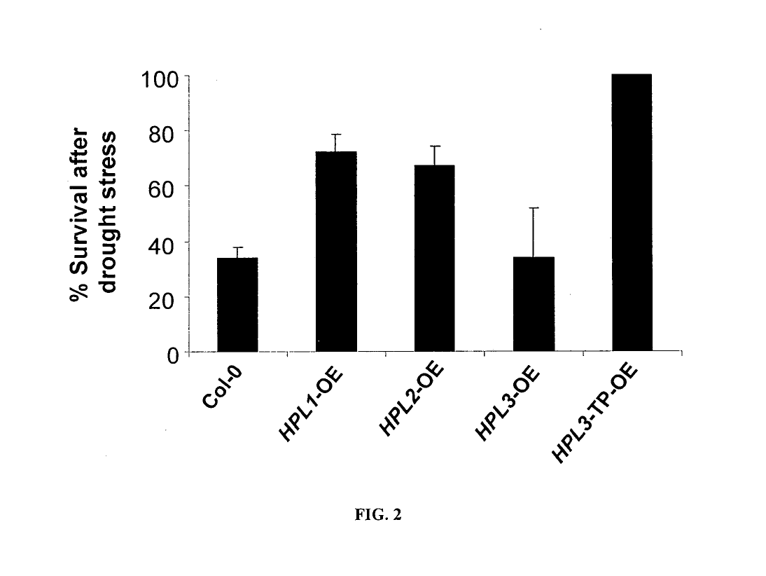

Enhanced tolerance of HPL1, HPL2 and HPL3-TP (HPL3 minus the 15 amino acids of the plastid transit peptide with localization of the enzyme outside the plastid) lines to drought-stress was observed (p≦0.001, 0.004, and 0.001, respectively), as measured by the survival rate of plants after ten days of water withdrawal (FIG. 2). Col-0, a natural hpl null mutant, was used as a control. In contrast to the lines in which HPL was localized outside the plastid, survival of the HPL3 line (enzyme was localized in the plastid) did not differ from the Col-0 line. Plants were grown in individual pots containing the same amount of soil. All pots were watered with the same amount of water. When plants were 2.5 weeks old (all plants had 8-10 true leaves), water was withheld for 9 days, a time point at which about 50% of Col-0 plants looked dead. Subsequently plants were watered excessively and were left to recover for 5 days before further analysis. Three independent experiments were carried out. In each experiment, each line was represented by 10-14 plants.

### Example 5

### Micro-array Analysis of Arabidopsis Lines Expressing Rice HPL1 and HPL3

Gene expression levels was evaluated in leaves from Arabidopsis lines expressing rice HPL1 and rice HPL3 compared to Col-0. RNA was extracted from leaves of three-week old plants grown in a growth chamber under standard conditions (16-hours light/8-hours dark cycle at 22° C.). The three biological samples (HPL1, HPL3, Col-0) were run in duplicate using Arabidopsis chips from Agilent Technologies. While several differences were observed in gene expression levels between the HPL1 line and the HPL3 line compared to Col-0, of particular interest was the observation that several sequences associated with heat shock proteins and heat shock transcription factors were increased to a greater degree in the HPL1 line than in the HPL3 line (see TABLE 1).

TABLE 1
Examples of differentially expressed heat shock associated
proteins in the HPL1 line and the HPL3 line compared to Col-0.

| | Fold | Fold |
|---|---|---|
| | Change | Change |
| Brief Sequence Description | HPL1 | HPL3 |
| At2g26150: heat shock transcription factor family | 8.5 | 2.22 |
| protein | | |
| At5g51440: small heat shock protein (HSP23.5-M) | 8.37 | 2.15 |
| At3g12580: heat shock protein 70 | 5.65 | 1.8 |
| At1g07400: 17.8 kDa class I heat shock protein | 5.23 | 1.68 |
| At2g20560: DNAJ heat shock family protein | 2.82 | 1.48 |
| SP\|Q9UDY4 | | |
| At1g74310: heat shock protein 101 (HSP101) | 2.74 | ND |
| At5g37670: 15.7 kDa class I-related small heat shock | 2.44 | 1.41 |
| protein-like (HSP15.7-CI) | | |
| At4g11660: heat shock transcription factor 7 (HSTF7) | 2.24 | 1.33 |
| At3g14200: DNAJ heat shock N-terminal domain- | 2 | ND |
| containing protein | | |
| At1g56410: heat shock cognate 70 kDa protein | 1.95 | ND |

One of these sequences, heat shock protein 101, was upregulated 2.74-fold in HPL1 compared to Col-0 but unchanged (ND) compared to Col-0 in the HPL3 line. Heat shock protein 101 has been shown recently to play an important role in conferring tolerance to heat (Tonsor et al., Mol. Ecol. 17(6):1614-1626, 2008). These data provide evidence that overexpression of HPL genes outside the plastid may provide protection against damage due to increasing temperature leading to enhanced heat tolerance.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

### Informal Sequence Listing

| (HPL1, AK105964) | |
|---|---|
| SEQ ID NO: 1 | |
| 1 | gtggctgtga cgatccgaca cctgcacgct agtacgtagt gcgtatacgt agccagtacc |
| 61 | ctactcccgt ccatggcgcc gccgcgagcc aactccggcg acggtaacga cggcgccgtc |
| 121 | ggagggcaga gcaagctctc gccgtcgggc ctgctgatac gcgagattcc gggcggctac |
| 181 | ggcgtgccct tcctctcgcc gctgcgcgac cgcctcgact actattactt ccagggcgcc |
| 241 | gacgagttct tccgctcacg cgtcgcccgc cacggcggcg ccaccgtgct ccgcgtcaac |
| 301 | atgccgcccg gccccttcct cgccggcgac ccccgcgtcg tcgccctcct cgacgcgcgc |
| 361 | agcttccgcg tcctcctcga cgactccatg gtggacaagg ccgacacgct cgacggcacc |
| 421 | ttcatgccgt cgctcgcgct cttcggcggc caccgcccgc tcgccttcct cgacgccgcc |
| 481 | gaccctcgcc acgccaagat caagcgcgtc gtgatgtcgc tcgccgcggc gaggatgcac |
| 541 | cacgtcgcgc cggcgttccg cgccgccttc gccgccatgt tcgacgaggt cgacgccggc |
| 601 | ctcgtcgccg gcggccccgt cgagttcaac aagctcaaca tgcggtacat gctcgacttc |
| 661 | acctgcgccg cgctgttcgg cggcgcgccg ccgagcaagg ccatgggcga cgctgccgtg |
| 721 | acgaaggcgg tgaagtggct catcttccag cttcacccgc tcgccagcaa ggtcgtcaag |
| 781 | ccgtggccgc tggaggacct cctcctccac accttccgcc tgccgccgtt cctggtgcgc |
| 841 | cgcgagtacg gcgagatcac ggcgtacttc gccgccgccg ccgcggccat cctcgacgac |
| 901 | gccgagaaga accacccggg aatcccgcgc gacgagctcc tccacaacct cgtgttcgtc |
| 961 | gccgtcttca acgcctacgg cggcttcaag atcttcctgc cacacatcgt caagtggctc |
| 1021 | gcccgcgccg gcccggagct ccacgccaag ctagcctccg aggtccgcgc cgccgcgccc |
| 1081 | gccggcggcg gcgagatcac catctccgcc gtggagaagg agatgccgct ggtgaagtcg |
| 1141 | gtggtgtggg aggcgctgcg catgaacccg ccggtggagt tccagtacgg gcgcgcgcgg |
| 1201 | cgcgacatgg tcgtcgagag ccacgacgcg gcgtacgagg tccgcaaggg ggagctgctg |
| 1261 | ttcgggtacc agccgctcgc cacccgcgac gagaaggtgt tcgaccgcgc cggcgagttc |
| 1321 | gtccccgacc ggttcgtctc cggcgccgga agcgccgccc ggccgctgct ggagcacgtg |
| 1381 | gtgtggtcga acgggccgga gaccgggacg ccatcggagg ggaacaagca gtgccccggg |
| 1441 | aaggacatgg tggtggcggt ggggcggctg atggtggcgg ggctgttccg gcggtacgac |
| 1501 | acgttcgccg ccgacgtgga ggagctgccg cttgagccgg tggtcacgtt cacgtcgctg |
| 1561 | acccgcgccg ccgacggcga cggcgccgcg cggcgcggag tataatagtg tcaagcggcg |
| 1621 | gcgcgcgtga gcggcgagtg ttggtgcggc gacgacgctg tccatgcatg gtcgctgtca |
| 1681 | gttggtcaga tttgcatgga tttctttttt ctttgaccta aaaaaattgg gaaaaaggtg |
| 1741 | tactttcgcg tgcttgtggg ggcaggttct taagtatagg gattcggttt gtcattgtgt |
| 1801 | gaagttcaat acgatgtttg aagttgaata aaattatgtg cgttcctcgt ggtttt |
| SEQ ID NO: 2 | |
| MAPPRANSGDGNDGAVGGQSKLSPSGLLIREIPGGYGVPFLSPLRDRLDYYYFQGADEFFRSRVARHGGATVLRVN | |
| MPPGPFLAGDPRVVALLDARSFRVLLDDSMVDKADTLDGTFMPSLALFGGHRPLAFLDAADPRHAKIKRVVMSLAA | |
| ARMHHVAPAFRAAFAAMFDEVDAGLVAGGPVEFNKLNMRYMLDFTCAALFGGAPPSKAMGDAAVTKAVKWLIFQLH | |
| PLASKVVKPWPLEDLLLHTFRLPPFLVRREYGEITAYFAAAAAAILDDAEKNHPGIPRDELLHNLVFVAVFNAYGG | |
| FKIFLPHIVKWLARAGPELHAKLASEVRAAAPAGGGEITISAVEKEMPLVKSVVWEALRMNPPVEFQYGRARRDMV | |
| VESHDAAYEVRKGELLFGYQPLATRDEKVFDRAGEFVPDRFVSGAGSAARPLLEHVVWSNGPETGTPSEGNKQCPG | |
| KDMVVAVGRLMVAGLFRRYDTFAADVEELPLEPVVTFTSLTRAADGDGAARRGV | |
| (HPL2, AK107161) | |
| SEQ ID NO: 3 | |
| 1 | ctcctcgaac caacccaaca caacacttgc acttgcacta cgtactctca tttcatccgc |
| 61 | tcccggccgg caatggcgcc accgccagtg aactccggcg acgccgccgc cgccgccacg |
| 121 | ggagagaaga gcaagctctc gccgtcgggc ctccccatac gcgagatacc cggcggctac |
| 181 | ggcgtgccct tcttctcgcc gctgcgcgac cgcctcgact acttctactt ccagggcgcc |
| 241 | gaggagtact tccgatcacg cgtcgcccgc cacggcggcg ccaccgtgct ccgcgtcaac |
| 301 | atgccgcccg gccccttcat ctccggcaac ccccgcgtcg tcgccctcct cgacgcgcgc |
| 361 | agcttccgcg tcctcctcga cgactccatg gtggacaagg ccgacacgct cgacggcacc |
| 421 | tacatgccgt cgcgcgcgct cttcggcggc caccgcccgc tcgccttcct cgacgccgcc |
| 481 | gacccgcgcc acgccaagat caagcgcgtc gtgatgtcgc tcgccgccgc gcggatgcac |
| 541 | cacgtcgcgc cggcgttccg cgccgccttt gccgccatgt tcgacgccgt cgaggccggc |
| 601 | ctcggcgccg ccgtcgagtt caacaagctc aacatgaggt acatgctcga cttcacctgc |
| 661 | gccgcgctgt tcggcggcga gccgccgagc aaggtggtcg gcgacggcgc cgtgacgaag |
| 721 | gccatggcgt ggctcgcgtt ccagctgcac ccgatcgcga gcaaggtcgt caagccatgg |
| 781 | ccgctcgagg agctactcct gcacaccttc tccctgccgc cgttcctggt gcggcgtggc |
| 841 | tacgccgacc tgaaggcgta cttcgccgac gccgccgcgg ccgtcctcga cgacgccgag |
| 901 | aagagccaca cgggaatccc gcgcgacgag ctcctcgaca accttgtgtt cgtcgccatt |
| 961 | ttcaacgcct tcggcggctt caagatcttc ctgccacaca tcgtcaagtg gctcgcccgc |
| 1021 | gccggcccgg agctccacgc caagcttgcc accgaggtcc gcgccaccgt gcccaccggc |
| 1081 | gaggacgacg gcatcaccct cgccgccgtc gagcggatgc cgctggtgaa gtcggtggtg |
| 1141 | tgggaggcgc tgcgcatgaa cccgccggtg gagttccagt acggccacgc gcggcgcgac |
| 1201 | atggtggtcg agagccacga cgcggcgtac gaggtgcgca agggggagat gctgttcggc |
| 1261 | taccagccgc tcgccacccg cgacgagaag gtgttcgacc gcgccggcga gttcgtcgcc |
| 1321 | gaccggttcg tcgccggcgg cgccgccggc gaccggccgc tgctggagca cgtggtgtgg |
| 1381 | tcgaacgggc cggagacgag ggcgccatcg gaggggaaca agcagtgccc cgggaaggac |
| 1441 | atggtggtgg cggtggggcg gctgatggtg gcggagctgt tccggcggta cgacacgttc |
| 1501 | gccgccgacg tggtggaggc gccggtggag ccggtggtga cgttcacgtc gctgacacgg |
| 1561 | gcgtcgtcgg gatagcacgc acgtcgacgt cacgtgcgcg ccgtgctgtg atttagtact |
| 1621 | gtactaggtt ggtggatgtt ttaattgcgt ggttaattat taatcacgca taaagtatta |
| 1681 | atcatgtttt atcatctaac aacaatgaaa atattaatca t |
| SEQ ID NO: 4 | |
| MAPPPVNSGDAAAAATGEKSKLSPSGLPIREIPGGYGVPFFSPLRDRLDYFYFQGAEEYFRSRVARHGGATVLRVN | |
| MPPGPFISGNPRVVALLDARSFRVLLDDSMVDKADTLDGTYMPSRALFGGHRPLAFLDAADPRHAKIKRVVMSLAA | |
| ARMHHVAPAFRAAFAAMFDAVEAGLGAAVEFNKLNMRYMLDFTCAALFGGEPPSKVVGDGAVTKAMAWLAFQLHPI | |
| ASKVVKPWPLEELLLHTFSLPPFLVRRGYADLKAYFADAAAAVLDDAEKSHTGIPRDELLDNLVFVAIFNAFGGFK | |
| IFLPHIVKWLARAGPELHAKLATEVRATVPTGEDDGITLAAVERMPLVKSVVWEALRMNPPVEFQYGHARRDMVVE | |
| SHDAAYEVRKGEMLFGYQPLATRDEKVFDRAGEFVADRFVAGGAAGDRPLLEHVVWSNGPETRAPSEGNKQCPGKD | |
| MVVAVGRLMVAELFRRYDTFAADVVEAPVEPVVTFTSLTRASSG | |
| (HPL3, AY340220) | |
| SEQ ID NO: 5 | |
| 1 | tagagtcagt gtcataacgc aagctaccac acgtagctga taagtccgat cgtcgccgcg |
| 61 | cgccgcgcca tggtgccgtc gttcccgcag ccggccagtg cggcggcggc gacgcggcca |
| 121 | ataccgggga gctacggccc gccgctgctc ggcccgctcc gcgaccgcct cgactacttc |
| 181 | tggttccagg gccccgacga cttcttccgc cgccgcgccg ccgaccacaa gagcaccgtg |
| 241 | ttccgcgcca acatcccgcc caccttcccc ttcttcctcg gcgtcgaccc gcgcgtcgtc |
| 301 | gccgtcgttg atgccgccgc cttcaccgcg ctcttcgacc cggccctcgt cgacaagcgc |
| 361 | gacgtcctca tcggccccta cgtccccagc ctcgccttca cccgcggcac ccgcgtcggc |
| 421 | gtctacctcg acacccagga ccccgaccac gcccgcacca aggccttctc catcgacctc |
| 481 | ctccgccgcg ccgcccgcaa ctgggccgcc gagctccgcg ccgccgtcga cgacatgctc |
| 541 | gccgccgtcg aggaagacct caacagggcc cctgaccccg ccgccgcctc cgccagctac |
| 601 | ctcatcccgc tccagaagtg catcttccgc ttcctctgca aggcgctcgt cggcgccgac |
| 661 | ccggcggcgg acggcctcgt cgaccgcttc ggcgtgtaca tcctcgacgt gtggctggcg |
| 721 | ttgcagctgg tgccgacgca gaaggtgggc gtcatcccgc agccgctgga ggagctcctg |
| 781 | ctccactcct tcccgctgcc gtcgttcgtc gtcaagcccg ggtacgacct cctctaccgc |
| 841 | ttcgtggaga agcacggcgc cgccgccgtg tccatcgctg agaaggagca cggcatcagc |
| 901 | aaggaggagg ccatcaacaa catcctcttc gtgctcggct tcaacgcgtt cggcggcttc |
| 961 | tcggtgttcc tgccgttcct ggtcatggag gtcggcaagc ccggccggga agacctgcgg |
| 1021 | cggcggctgc gggaggaggt gcgccgcgtg ctgggcggcg gcgacggcgg cgaggccggg |
| 1081 | ttcgcggcgg tgagggagat ggcgctggtg cggtcgacgg tgtacgaggt gctccggatg |
| 1141 | cagccgccgg tgccgctgca gttcgggcgg gcgcggcgag acttcgtgct gcggtcgcac |
| 1201 | ggcggcgcgg cgtacgaggt gggcaagggc gagctgctgt gcgggtacca gccgctggcc |
| 1261 | atgcgcgacc cggcggtgtt cgaccggccg gaggagttcg cgccggagag gttcctcggc |
| 1321 | gacgacggcg aggcgctgct gcagtacgtg tactggtcca acgggccgga gaccggcgag |
| 1381 | ccgtcgccgg ggaacaaaca gtgtgccgcc aaggaggtgg tcgtcgccac cgcgtgcatg |
| 1441 | ctcgtcgccg agcttttccg gcggtacgac gacttcgaat gcgacggcac ctccttcacc |
| 1501 | aagctcgaca agcgggagct cactcccagc taagctttgc tgccgccatt ctctcactcg |
| 1561 | atctccatgc acatatgcat gaagaaatta attaaattca agttgctagc tccatttttt |
| 1621 | ctctttgagc tgctgataaa aaaacatctc tattcttctg tgcaataagc caataattaa |
| 1681 | gcattaatca gagcgtacaa gtaaaaattg ttttcactgt tttatgtgga t |

underlined sequences are deleted to prevent transport of encoded polypeptide
into the plastid
SEQ ID NO: 6
MVPSFPQPASAAAATRPIPGSYGPPLLGPLRDRLDYFWFQGPDDFFRRRAADHKSTVFRANIPPTFPFFLGVDPRV
VAVVDAAAFTALFDPALVDKRDVLIGPYVPSLAFTRGTRVGVYLDTQDPDHARTKAFSIDLLRRAARNWAAELRAA
VDDMLAAVEEDLNRAPDPAAASASYLIPLQKCIFRFLCKALVGADPAADGLVDRFGVYILDVWLALQLVPTQKVGV
IPQPLEELLLHSFPLPSFVVKPGYDLLYRFVEKHGAAAVSIAEKEHGISKEEAINNILFVLGFNAFGGFSVFLPFL
VMEVGKPGREDLRRRLREEVRRVLGGGDGGEAGFAAVREMALVRSTVYEVLRMQPPVPLQFGRARRDFVLRSHGGA
AYEVGKGELLCGYQPLAMRDPAVFDRPEEFAPERFLGDDGEALLQYVYWSNGPETGEPSPGNKQCAAKEVVVATAC
MLVAELFRRYDDFECDGTSFTKLDKRELTPS
underlined sequences are deleted to prevent transport of polypeptide into
the plastid

1. A method of preparing a plant tolerant to abiotic stress, the method comprising:
(a) introducing into a population of plants a recombinant expression cassette comprising a hydroperoxide lyase (HPL) polynucleotide encoding a HPL enzyme; and
(b) selecting a plant that is tolerant to abiotic stress;
wherein the HPL enzyme comprises (L/I)-(F/C)-G-(Y/F)-(Q/R)-(P/K) and (N/D)-K-(Q/D-C-(A/P)-(G/A)-K-(D/N).

2. The method of claim 1, wherein the HPL enzyme is localized outside the plastid when expressed in the population of plants.

3. The method of claim 1, wherein the HPL enzyme recognizes 9-hydroperoxy-octadecatrienoic acid (9-HPOT) or 9-hydroperoxy-octadecadienoic acid (9-HPOD).

4. The method of claim 1, wherein the HPL enzyme recognizes 13-hydroperoxy-octadecatrienoic acid (13-HPOT) or 13-hydroperoxy-octadecadienoic acid (13-HPOD).

5. The method of claim 1, wherein the HPL enzyme is localized outside the plastid when expressed in the population of plants, and wherein the HPL enzyme recognizes 9-hydroperoxy-octadecatrienoic acid (9-HPOT) or 9-hydroperoxy-octadecadienoic acid (9-HPOD).

6. The method of claim 5, wherein the HPL enzyme further recognizes 13-hydroperoxy-octadecatrienoic acid (13-HPOT) or 13-hydroperoxy-octadecadienoic acid (13-HPOD).

7. The method of claim 1, wherein the HPL enzyme has an amino acid sequence at least 90% identical to SEQ ID NO. 2, 4 or 6.

8. A method of preparing a plant tolerant to abiotic stress, the method comprising:
(a) introducing into a population of plants a recombinant expression cassette comprising a hydroperoxide lyase (HPL) polynucleotide encoding a HPL enzyme wherein the HPL enzyme has an amino acid sequence at least 90% identical to SEQ ID NO. 2, 4 or 6; and
(b) selecting a plant that is tolerant to abiotic stress,
wherein the HPL enzyme is localized outside the plastid when expressed in the population of plants.

9. The method of claim 1, wherein the step of introducing is carried out by a sexual cross.

10. The method of claim 1, wherein the step of introducing is carried out using micro-projectile bombardment.

11. The method of claim 1, wherein the HPL polynucleotide is SEQ ID NO. 1, 3 or 5.

12. The method of claim 1, wherein the plant is Oryza sativa.

13. The method of claim 1, wherein the abiotic stress is drought.

14. The method of claim 1, wherein the abiotic stress is salt.

15. The method of claim 1, wherein the HPL polynucleotide is operably linked to a promoter.

16. The method of claim 15, wherein the promoter is constitutive or inducible.

17. A transgenic plant comprising a recombinant expression cassette, wherein the recombinant expression cassette comprises a HPL polynucleotide encoding a HPL enzyme having an amino acid sequence at least 90% identical to SEQ ID NO. 2, 4 or 6, with a proviso that the plant is not Arabidopsis.

18. The transgenic plant of claim 17, wherein the plant is Oryza sativa.

19. A transgenic seed from the transgenic plant of claim 18.

20. The transgenic seed of claim 19, wherein the seed is a transgenic Oryza sativa seed.


SEQUENCE LISTING (21 sequences)

SEQ ID NO: 1 - DNA, 1856 bp, Oryza sativa
  rice hydroperoxide lyase (HPL1)
gtggctgtgacgatccgacacctgcacgctagtacgtagtgcgtatacgtagccagtaccctactcccgtccatggcgccgccgcgagccaactccggcgacggtaacgacggcgccgtcggagggcagagcaagctctcgccgtcgggcctgctgatacgcgagattccgggcggctacggcgtgcccttcctctcgccgctgcgcgaccgcctcgactactattacttccagggcgccgacgagttcttccgctcacgcgtcgcccgccacggcggcgccaccgtgctccgcgtcaacatgccgcccggccccttcctcgccggcgacccccgcgtcgtcgccctcctcgacgcgcgcagcttccgcgtcctcctcgacgactccatggtggacaaggccgacacgctcgacggcaccttcatgccgtcgctcgcgctcttcggcggccaccgcccgctcgccttcctcgacgccgccgaccctcgccacgccaagatcaagcgcgtcgtgatgtcgctcgccgcggcgaggatgcaccacgtcgcgccggcgttccgcgccgccttcgccgccatgttcgacgaggtcgacgccggcctcgtcgccggcggccccgtcgagttcaacaagctcaacatgcggtacatgctcgacttcacctgcgccgcgctgttcggcggcgcgccgccgagcaaggccatgggcgacgctgccgtgacgaaggcggtgaagtggctcatcttccagcttcacccgctcgccagcaaggtcgtcaagccgtggccgctggaggacctcctcctccacaccttccgcctgccgccgttcctggtgcgccgcgagtacggcgagatcacggcgtacttcgccgccgccgccgcggccatcctcgacgacgccgagaagaaccacccgggaatcccgcgcgacgagctcctccacaacctcgtgttcgtcgccgtcttcaacgcctacggcggcttcaagatcttcctgccacacatcgtcaagtggctcgcccgcgccggcccggagctccacgccaagctagcctccgaggtccgcgccgccgcgcccgccggcggcggcgagatcaccatctccgccgtggagaaggagatgccgctggtgaagtcggtggtgtgggaggcgctgcgcatgaacccgccggtggagttccagtacgggcgcgcgcggcgcgacatggtcgtcgagagccacgacgcggcgtacgaggtccgcaagggggagctgctgttcgggtaccagccgctcgccacccgcgacgagaaggtgttcgaccgcgccggcgagttcgtccccgaccggttcgtctccggcgccggaagcgccgcccggccgctgctggagcacgtggtgtggtcgaacgggccggagaccgggacgccatcggaggggaacaagcagtgccccgggaaggacatggtggtggcggtggggcggctgatggtggcggggctgttccggcggtacgacacgttcgccgccgacgtggaggagctgccgcttgagccggtggtcacgttcacgtcgctgacccgcgccgccgacggcgacggcgccgcgcggcgcggagtataatagtgtcaagcggcggcgcgcgtgagcggcgagtgttggtgcggcgacgacgctgtccatgcatggtcgctgtcagttggtcagatttgcatggatttcttttttctttgacctaaaaaaattgggaaaaaggtgtactttcgcgtgcttgtgggggcaggttcttaagtatagggattcggtttgtcattgtgtgaagttcaatacgatgtttgaagttgaataaaattatgtgcgttcctcgtggtttt

SEQ ID NO: 2 - protein, 510 aa, Oryza sativa
  rice hydroperoxide lyase (HPL1)
MAPPRANSGDGNDGAVGGQSKLSPSGLLIREIPGGYGVPFLSPLRDRLDYYYFQGADEFFRSRVARHGGATVLRVNMPPGPFLAGDPRVVALLDARSFRVLLDDSMVDKADTLDGTFMPSLALFGGHRPLAFLDAADPRHAKIKRVVMSLAAARMHHVAPAFRAAFAAMFDEVDAGLVAGGPVEFNKLNMRYMLDFTCAALFGGAPPSKAMGDAAVTKAVKWLIFQLHPLASKVVKPWPLEDLLLHTFRLPPFLVRREYGEITAYFAAAAAAILDDAEKNHPGIPRDELLHNLVFVAVFNAYGGFKIFLPHIVKWLARAGPELHAKLASEVRAAAPAGGGEITISAVEKEMPLVKSVVWEALRMNPPVEFQYGRARRDMVVESHDAAYEVRKGELLFGYQPLATRDEKVFDRAGEFVPDRFVSGAGSAARPLLEHVVWSNGPETGTPSEGNKQCPGKDMVVAVGRLMVAGLFRRYDTFAADVEELPLEPVVTFTSLTRAADGDGAARRGV

SEQ ID NO: 3 - DNA, 1721 bp, Oryza sativa
  rice hydroperoxide lyase (HPL2)
ctcctcgaaccaacccaacacaacacttgcacttgcactacgtactctcatttcatccgctcccggccggcaatggcgccaccgccagtgaactccggcgacgccgccgccgccgccacgggagagaagagcaagctctcgccgtcgggcctccccatacgcgagatacccggcggctacggcgtgcccttcttctcgccgctgcgcgaccgcctcgactacttctacttccagggcgccgaggagtacttccgatcacgcgtcgcccgccacggcggcgccaccgtgctccgcgtcaacatgccgcccggccccttcatctccggcaacccccgcgtcgtcgccctcctcgacgcgcgcagcttccgcgtcctcctcgacgactccatggtggacaaggccgacacgctcgacggcacctacatgccgtcgcgcgcgctcttcggcggccaccgcccgctcgccttcctcgacgccgccgacccgcgccacgccaagatcaagcgcgtcgtgatgtcgctcgccgccgcgcggatgcaccacgtcgcgccggcgttccgcgccgcctttgccgccatgttcgacgccgtcgaggccggcctcggcgccgccgtcgagttcaacaagctcaacatgaggtacatgctcgacttcacctgcgccgcgctgttcggcggcgagccgccgagcaaggtggtcggcgacggcgccgtgacgaaggccatggcgtggctcgcgttccagctgcacccgatcgcgagcaaggtcgtcaagccatggccgctcgaggagctactcctgcacaccttctccctgccgccgttcctggtgcggcgtggctacgccgacctgaaggcgtacttcgccgacgccgccgcggccgtcctcgacgacgccgagaagagccacacgggaatcccgcgcgacgagctcctcgacaaccttgtgttcgtcgccattttcaacgccttcggcggcttcaagatcttcctgccacacatcgtcaagtggctcgcccgcgccggcccggagctccacgccaagcttgccaccgaggtccgcgccaccgtgcccaccggcgaggacgacggcatcaccctcgccgccgtcgagcggatgccgctggtgaagtcggtggtgtgggaggcgctgcgcatgaacccgccggtggagttccagtacggccacgcgcggcgcgacatggtggtcgagagccacgacgcggcgtacgaggtgcgcaagggggagatgctgttcggctaccagccgctcgccacccgcgacgagaaggtgttcgaccgcgccggcgagttcgtcgccgaccggttcgtcgccggcggcgccgccggcgaccggccgctgctggagcacgtggtgtggtcgaacgggccggagacgagggcgccatcggaggggaacaagcagtgccccgggaaggacatggtggtggcggtggggcggctgatggtggcggagctgttccggcggtacgacacgttcgccgccgacgtggtggaggcgccggtggagccggtggtgacgttcacgtcgctgacacgggcgtcgtcgggatagcacgcacgtcgacgtcacgtgcgcgccgtgctgtgatttagtactgtactaggttggtggatgttttaattgcgtggttaattattaatcacgcataaagtattaatcatgttttatcatctaacaacaatgaaaatattaatcat

SEQ ID NO: 4 - protein, 500 aa, Oryza sativa
  rice hydroperoxide lyase (HPL2)
MAPPPVNSGDAAAAATGEKSKLSPSGLPIREIPGGYGVPFFSPLRDRLDYFYFQGAEEYFRSRVARHGGATVLRVNMPPGPFISGNPRVVALLDARSFRVLLDDSMVDKADTLDGTYMPSRALFGGHRPLAFLDAADPRHAKIKRVVMSLAAARMHHVAPAFRAAFAAMFDAVEAGLGAAVEFNKLNMRYMLDFTCAALFGGEPPSKVVGDGAVTKAMAWLAFQLHPIASKVVKPWPLEELLLHTFSLPPFLVRRGYADLKAYFADAAAAVLDDAEKSHTGIPRDELLDNLVFVAIFNAFGGFKIFLPHIVKWLARAGPELHAKLATEVRATVPTGEDDGITLAAVERMPLVKSVVWEALRMNPPVEFQYGHARRDMVVESHDAAYEVRKGEMLFGYQPLATRDEKVFDRAGEFVADRFVAGGAAGDRPLLEHVVWSNGPETRAPSEGNKQCPGKDMVVAVGRLMVAELFRRYDTFAADVVEAPVEPVVTFTSLTRASSG

SEQ ID NO: 5 - DNA, 1731 bp, Oryza sativa
  rice hydroperoxide lyase (HPL3)
tagagtcagtgtcataacgcaagctaccacacgtagctgataagtccgatcgtcgccgcgcgccgcgccatggtgccgtcgttcccgcagccggccagtgcggcggcggcgacgcggccaataccggggagctacggcccgccgctgctcggcccgctccgcgaccgcctcgactacttctggttccagggccccgacgacttcttccgccgccgcgccgccgaccacaagagcaccgtgttccgcgccaacatcccgcccaccttccccttcttcctcggcgtcgacccgcgcgtcgtcgccgtcgttgatgccgccgccttcaccgcgctcttcgacccggccctcgtcgacaagcgcgacgtcctcatcggcccctacgtccccagcctcgccttcacccgcggcacccgcgtcggcgtctacctcgacacccaggaccccgaccacgcccgcaccaaggccttctccatcgacctcctccgccgcgccgcccgcaactgggccgccgagctccgcgccgccgtcgacgacatgctcgccgccgtcgaggaagacctcaacagggcccctgaccccgccgccgcctccgccagctacctcatcccgctccagaagtgcatcttccgcttcctctgcaaggcgctcgtcggcgccgacccggcggcggacggcctcgtcgaccgcttcggcgtgtacatcctcgacgtgtggctggcgttgcagctggtgccgacgcagaaggtgggcgtcatcccgcagccgctggaggagctcctgctccactccttcccgctgccgtcgttcgtcgtcaagcccgggtacgacctcctctaccgcttcgtggagaagcacggcgccgccgccgtgtccatcgctgagaaggagcacggcatcagcaaggaggaggccatcaacaacatcctcttcgtgctcggcttcaacgcgttcggcggcttctcggtgttcctgccgttcctggtcatggaggtcggcaagcccggccgggaagacctgcggcggcggctgcgggaggaggtgcgccgcgtgctgggcggcggcgacggcggcgaggccgggttcgcggcggtgagggagatggcgctggtgcggtcgacggtgtacgaggtgctccggatgcagccgccggtgccgctgcagttcgggcgggcgcggcgagacttcgtgctgcggtcgcacggcggcgcggcgtacgaggtgggcaagggcgagctgctgtgcgggtaccagccgctggccatgcgcgacccggcggtgttcgaccggccggaggagttcgcgccggagaggttcctcggcgacgacggcgaggcgctgctgcagtacgtgtactggtccaacgggccggagaccggcgagccgtcgccggggaacaaacagtgtgccgccaaggaggtggtcgtcgccaccgcgtgcatgctcgtcgccgagcttttccggcggtacgacgacttcgaatgcgacggcacctccttcaccaagctcgacaagcgggagctcactcccagctaagctttgctgccgccattctctcactcgatctccatgcacatatgcatgaagaaattaattaaattcaagttgctagctccattttttctctttgagctgctgataaaaaaacatctctattcttctgtgcaataagccaataattaagcattaatcagagcgtacaagtaaaaattgttttcactgttttatgtggat

SEQ ID NO: 6 - protein, 487 aa, Oryza sativa
  rice hydroperoxide lyase (HPL3)
MVPSFPQPASAAAATRPIPGSYGPPLLGPLRDRLDYFWFQGPDDFFRRRAADHKSTVFRANIPPTFPFFLGVDPRVVAVVDAAAFTALFDPALVDKRDVLIGPYVPSLAFTRGTRVGVYLDTQDPDHARTKAFSIDLLRRAARNWAAELRAAVDDMLAAVEEDLNRAPDPAAASASYLIPLQKCIFRFLCKALVGADPAADGLVDRFGVYILDVWLALQLVPTQKVGVIPQPLEELLLHSFPLPSFVVKPGYDLLYRFVEKHGAAAVSIAEKEHGISKEEAINNILFVLGFNAFGGFSVFLPFLVMEVGKPGREDLRRRLREEVRRVLGGGDGGEAGFAAVREMALVRSTVYEVLRMQPPVPLQFGRARRDFVLRSHGGAAYEVGKGELLCGYQPLAMRDPAVFDRPEEFAPERFLGDDGEALLQYVYWSNGPETGEPSPGNKQCAAKEVVVATACMLVAELFRRYDDFECDGTSFTKLDKRELTPS

SEQ ID NO: 7 - protein, 8 aa, Artificial Sequence
  synthetic HPL consensus sequence
BKXCXXKB

SEQ ID NO: 8 - DNA, 40 bp, Artificial Sequence
  synthetic gene-specific PCR amplification OsHPL1 forward primer oligonucleotide
atagatatcgcatgcatggcgccgccgcgagccaactccg

SEQ ID NO: 9 - DNA, 40 bp, Artificial Sequence
  synthetic gene-specific PCR amplification OsHPL1 reverse primer oligonucleotide
atatacgtactgcagcgcgcgccgccgcttgacactatta

SEQ ID NO: 10 - DNA, 40 bp, Artificial Sequence
  synthetic gene-specific PCR amplification OsHPL2 forward primer oligonucleotide
atagatatcgcatgcatggcgccaccgccagtgaactccg

SEQ ID NO: 11 - DNA, 40 bp, Artificial Sequence
  synthetic gene-specific PCR amplification OsHPL2 reverse primer oligonucleotide
atatacgtactgcaggcacgtgacgtcgacgtgcgtgcta

SEQ ID NO: 12 - DNA, 40 bp, Artificial Sequence
  synthetic gene-specific PCR amplification OsHPL3 forward primer oligonucleotide
atagatatcgcatgcatggtgccgtcgttcccgcagccgg

SEQ ID NO: 13 - DNA, 40 bp, Artificial Sequence
  synthetic gene-specific PCR amplification OsHPL3 reverse primer oligonucleotide
atatacgtactgcaggagagaatggcggcagcaaagctta

SEQ ID NO: 14 - DNA, 25 bp, Artificial Sequence
  synthetic PCR amplification OsHPL1 forward primer for green fluorescent protein (GFP) fusions
atagaattcatggcgccgccgcgag

SEQ ID NO: 15 - DNA, 27 bp, Artificial Sequence
  synthetic PCR amplification OsHPL1 reverse primer for green fluorescent protein (GFP) fusions
ataggatccgctactccgcgccgcgcg

SEQ ID NO: 16 - DNA, 26 bp, Artificial Sequence
  synthetic PCR amplification OsHPL2 forward primer for green fluorescent protein (GFP) fusions
atagaattcatggcgccaccgccagt

SEQ ID NO: 17 - DNA, 27 bp, Artificial Sequence
  synthetic PCR amplification OsHPL2 reverse primer for green fluorescent protein (GFP) fusions
ataggatccgctcccgacgacgcccgt

SEQ ID NO: 18 - DNA, 26 bp, Artificial Sequence
  synthetic PCR amplification OsHPL3 forward primer for green fluorescent protein (GFP) fusions
atagaattcatggtgccgtcgttccc

SEQ ID NO: 19 - DNA, 28 bp, Artificial Sequence
  synthetic PCR amplification OsHPL3 reverse primer for green fluorescent protein (GFP) fusions
ataggatccgcgctgggagtgagctccc

SEQ ID NO: 20 - DNA, 16 bp, Artificial Sequence
  synthetic PCR amplification forward primer OsHPL3-TP for HPL3 minus 15 amino acid N-terminal plastid transit peptide
ccggccaataccgggg

SEQ ID NO: 21 - DNA, 18 bp, Artificial Sequence
  synthetic PCR amplification reverse primer OsHPL3-TP for HPL3 minus 15 amino acid N-terminal plastid transit peptide
ttagctgggagtgagctc